(19) United States
(12) Patent Application Publication
(10) Pub. No.: US 2013/0123511 A1
(43) Pub. Date: May 16, 2013

(54) PROCESS FOR THE DIRECT PREPARATION OF MALIC ACID SALT OF SUNITINIB

(71) Applicants: Sudhir Singh Sanwal, Kangra (IN); Saridi Madhava Dileep Kumar, Gurgaon (IN); Swargam Sathyanarayana, Karim Nagar (IN); Rajesh Kumar Thaper, Jammu (IN); Mohan Prasad, Gurgaon (IN)

(72) Inventors: Sudhir Singh Sanwal, Kangra (IN); Saridi Madhava Dileep Kumar, Gurgaon (IN); Swargam Sathyanarayana, Karim Nagar (IN); Rajesh Kumar Thaper, Jammu (IN); Mohan Prasad, Gurgaon (IN)

(73) Assignee: RANBAXY LABORATORIES LIMITED, New Delhi, Delhi (IN)

(21) Appl. No.: 13/582,493

(22) PCT Filed: Feb. 25, 2011
(86) PCT No.: PCT/IB2011/050821

§ 371 Date: Dec. 5, 2012

(30) Foreign Application Priority Data
Mar. 4, 2010 (IN) 478/DEL/2010

(51) Int. Cl. C07D 403/06; C07C 51/41

(52) U.S. Cl. CPC C07D 403/06; C07C 51/412
USPC 548/468

(57) ABSTRACT

The present invention relates to a process for the direct preparation of malic acid salt of sunitinib.

### FIELD OF THE INVENTION

The present invention relates to a process for the direct preparation of malic acid salt of sunitinib.

### BACKGROUND OF THE INVENTION

Sunitinib is chemically described as N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide as represented by Formula I.

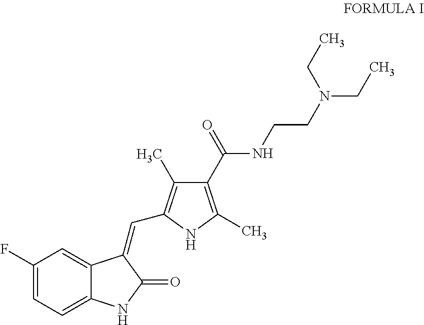 [SMILES: CCN(CC)CCNC(=O)c1c(C)[nH]c(/C=C2\C(=O)Nc3ccc(F)cc32)c1C]

Sunitinib is an oral multi-kinase inhibitor and is useful for the treatment of gastrointestinal stromal tumor and advanced renal cell carcinoma. Sunitinib is commercially available as L-malate salt, which is described chemically as butanedioic acid, hydroxy-, (2S)-, compound with N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide (1:1).

U.S. Pat. No. 7,125,905 describes a process for the preparation of sunitinib base wherein the process involves heating a mixture of N-[2-(diethylamino)ethyl]-5-formyl-2,4-dimethyl-1H-pyrrole-3-carboxamide of Formula II and 5-fluoro-1,3-dihydro-2H-indol-2-one of Formula III in the presence of ethanol and pyrrolidine at 78° C. for 3 hours. The mixture is cooled to room temperature and sunitinib is collected as a base by vacuum filtration.

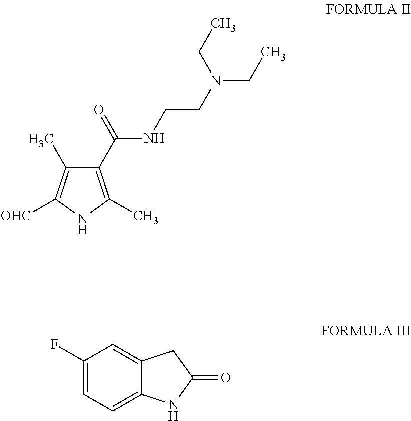 [SMILES: CCN(CC)CCNC(=O)c1c(C)[nH]c(C=O)c1C ; O=C1Cc2cc(F)ccc2N1]

U.S. Publication Nos. 2003/0069298 and 2007/0191458 describe the preparation of crystal Forms I and II of L-malic acid salt of sunitinib from sunitinib base. PCT Publication No. WO 2009/067686 describes processes for preparing crystalline forms of racemic sunitinib malate, sunitinib hemi-L-malate and compositions containing sunitinib base and L- or racemic malic acid from sunitinib base.

WO 2009/150523 describes processes for the preparation of L-malic acid salt of sunitinib, wherein the process involves preparation of L-malic acid salt of N-[2-(diethylamino)ethyl]-5-formyl-2,4-dimethyl-1H-pyrrole-3-carboxamide of Formula II and reacting the salt with 5-fluoro-1,3-dihydro-2H-indol-2-one of Formula III to obtain L-malic acid salt of sunitinib with 75.1% yield.

### SUMMARY OF THE INVENTION

The present inventors have developed a simple and efficient process for the preparation of the malic acid salt of sunitinib. The present process neither requires the preparation of malic acid salt of N-[2-(diethylamino)ethyl]-5-formyl-2,4-dimethyl-1H-pyrrole-3-carboxamide of Formula II nor does it require the conversion of sunitinib base into malic acid salt of sunitinib. The malic acid salt of sunitinib can be obtained by the present process with a yield of about 80% or above directly from the reaction mixture obtained after reacting N-[2-(diethylamino)ethyl]-5-formyl-2,4-dimethyl-1H-pyrrole-3-carboxamide of Formula II and 5-fluoro-1,3-dihydro-2H-indol-2-one of Formula III.

The term “malic acid salt of sunitinib” includes a combination of sunitinib and malic acid in any ratio between about 1:0.5 and about 1:1.5.

### DETAILED DESCRIPTION OF THE INVENTION

In one aspect of the present invention is provided a process for the direct preparation of the malic acid salt of sunitinib, wherein the process comprises:
a) reacting N-[2-(diethylamino)ethyl]-5-formyl-2,4-dimethyl-1H-pyrrole-3-carboxamide of Formula II with 5-fluoro-1,3-dihydro-2H-indol-2-one of Formula III in the presence of malic acid and a solvent; and
b) isolating the malic acid salt of sunitinib from the reaction mixture thereof.

N-[2-(Diethylamino)ethyl]-5-formyl-2,4-dimethyl-1H-pyrrole-3-carboxamide of Formula II may be prepared according to the method described in, for example, U.S. Pat. No. 7,125,905. N-[2-(Diethylamino)ethyl]-5-formyl-2,4-dimethyl-1H-pyrrole-3-carboxamide of Formula II is reacted with 5-fluoro-1,3-dihydro-2H-indol-2-one of Formula III in the presence of malic acid and a solvent. The reaction may be carried out, for example, by adding N-[2-(diethylamino)ethyl]-5-formyl-2,4-dimethyl-1H-pyrrole-3-carboxamide of Formula II, 5-fluoro-1,3-dihydro-2H-indol-2-one of Formula III and malic acid to the solvent or by adding solvent to N-[2-(diethylamino)ethyl]-5-formyl-2,4-dimethyl-1H-pyrrole-3-carboxamide of Formula II, 5-fluoro-1,3-dihydro-2H-indol-2-one of Formula III and malic acid. The addition may be carried out, for example, sequentially. The solvent may be water, an organic solvent, or a mixture thereof. The organic solvent may be an alkanol, for example, n-propanol, methanol, ethanol, isopropanol or n-butanol, an ester, for example, n-butyl acetate, isopropyl acetate, methyl acetate or ethyl acetate, a nitrile, for example, acetonitrile, an aromatic hydrocarbon, for example, toluene, a cyclic ether, for example, tetrahydrofuran, or a ketone, for example, acetone, or a mixture thereof. The malic may be L-malic acid, D-malic acid, or a mixture thereof. The reaction mixture may also contain a base. The base may be an organic amine, for example, pyrrolidine. The reaction may be carried out at a temperature of about the boiling point of the solvent. For example, the reaction may be carried out at about 75° C. to about 80° C. when ethanol is used as a solvent. The reaction may be carried out for about 10 minutes to about 10 hours, for example, about 2 hours to about 5 hours. The malic acid salt of sunitinib is isolated from the reaction mixture by filtration, decantation, solvent precipitation, solvent evaporation, layer separation, centrifugation or a combination thereof.

While the present invention has been described in terms of its specific embodiments, certain modifications and equivalents will be apparent to those skilled in the art and are intended to be included within the scope of the present invention.

### EXAMPLE

### Preparation of L-Malic Acid Salt of Sunitinib

N-[2-(diethylamino)ethyl]-5-formyl-2,4-dimethyl-1H-pyrrole-3-carboxamide (1.0 g), 5-Fluoro-1,3-dihydro-2H-indol-2-one (0.57 g), pyrrolidine (0.013 g) and L-malic acid (0.37 g) were added to absolute ethanol (15 ml) and the reaction mixture was stirred at 78° C. (internal temperature) for 3 hours. The reaction mixture was cooled to 20° C. to 25° C., filtered under vacuum, washed with absolute ethanol (10 ml) and dried under vacuum at 50° C. for 10 hours to 12 hours to obtain the title compound.

Percentage yield: 80%

Purity: 99.37%.

We claim:

1. A process for the direct preparation of malic acid salt of sunitinib, wherein the process comprises:
a) reacting N-[2-(diethylamino)ethyl]-5-formyl-2,4-dimethyl-1H-pyrrole-3-carboxamide of Formula II 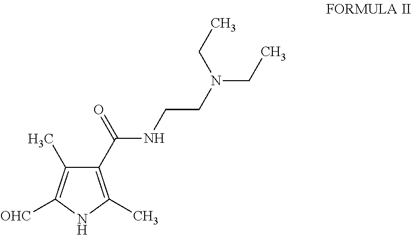 [SMILES: CCN(CC)CCNC(=O)c1c(C)[nH]c(C=O)c1C]
with 5-fluoro-1,3-dihydro-2H-indol-2-one of Formula III 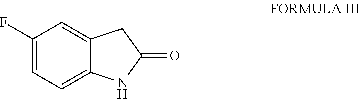 [SMILES: O=C1Cc2cc(F)ccc2N1]
in the presence of malic acid and a solvent; and
b) isolating malic acid salt of sunitinib from the reaction mixture thereof.

2. A process according to claim 1, wherein the solvent used in step a) is water, an organic solvent or a mixture thereof.

3. A process according to claim 2, wherein the organic solvent is alkanol, ester, nitrile, aromatic hydrocarbon, cyclic ether, ketone, or a mixture thereof.

4. A process according to claim 3, wherein the organic solvent is alkanol.

5. A process according to claim 4, wherein the alkanol is ethanol.

6. A process according to claim 1, wherein step a) is carried out in the presence of a base.

7. A process according to claim 6, wherein the base is organic amine.

8. A process according to claim 7, wherein the organic amine is pyrrolidine.

9. A process according to claim 1, wherein the malic acid used in step a) is L-malic acid or D-malic acid, or a mixture thereof.
